(19) United States
(12) Patent Application Publication
(10) Pub. No.: US 2021/0189311 A1
(43) Pub. Date: Jun. 24, 2021

(54) DEVICE AND METHOD FOR CULTURING CELLS IN A SHAKING OPERATION

(71) Applicant: AQUILA BIOLABS GmbH, Baesweiler (DE)

(72) Inventors: David Frank, Herzogenrath (DE); Konrad Herzog, Herzogenrath (DE)

(21) Appl. No.: 17/054,364

(22) PCT Filed: May 8, 2019
(86) PCT No.: PCT/EP2019/061768

§ 371 Date: Nov. 10, 2020

(30) Foreign Application Priority Data
May 16, 2018 (DE) 10 2018 003 910.3

(51) Int. Cl. C12M 1/24; C12M 1/04; C12M 1/34; C12M 3/06

(52) U.S. Cl. CPC C12M 23/08; C12M 23/24; C12M 41/42; C12M 27/16; C12M 41/34

(57) ABSTRACT

A device for culturing cells in a shaking operation with passive gas exchange, which includes at least one gas exchange opening, characterized in that the opening surface of the gas exchange opening is not oriented parallel with the movement vector of the shaking movement at at least one point in time of the shaking movement. Owing to the shaking movement and the ambient gas phase flowing around the outside of the device and the headspace gas phase flowing around the inside of the device, which flowing is associated with the shaking, the arrangement leads to the formation of a pressure gradient between the headspace gas phase and the ambient gas phase, which pressure gradient changes depending on the shaking movement and advantageously influences the gas exchange between the headspace gas phase and the ambient gas phase and the gas blending within the headspace.

### CROSS REFERENCE TO RELATED APPLICATIONS

This is a US national phase application under 37 C.F.R § 371 of PCT/EP2019/061768, filed 8 May 2019, which claims priority to German patent application no. 10 2018 003 910.3, filed 16 May 2018. Each application referred to in this paragraph is herein incorporated by reference in its entirety.

### TECHNICAL FIELD

The invention relates to a device for culturing cells in a shaking operation and also a method for the application thereof.

### BACKGROUND OF THE INVENTION

A plurality of different actively-gassed culturing systems from the group of stirred-tank fermenters are known to a person skilled in the art, which systems are gassed with sterile-filtered gas mixtures with regulation of pressure and volumetric flow, with the gas exchange between inflowing gas phase and culture liquid mainly taking place at the interfaces of the gas bubbles bubbling through the culture liquid. Compared to passively-gassed devices for culturing cells, active culturing systems are disadvantageously considerably more complex in terms of construction, handling, operation and maintenance. Therefore, no further reference will be made to these devices.

Passively-gassed devices for culturing cells are essentially based on the concept of a liquid container with differently-closeable filling openings at the top, via which the gas exchange with the ambient gas phase also takes place, such that the gassing and the handling of the culture liquid (filling, sampling, etc.) take place via the same opening. The mixing of such culturing devices occurs via the movement of the device in conjunction with the inertia and friction of the liquid and gas phases present in the device. Common devices that implement this concept include in particular a wide variety of culture flasks and bottles and also multi-well plates. Mixing takes place in particular on rocking, tumbling or orbital shakers, which can also be configured as incubators in order to set defined temperature, humidity and ambient gas phase conditions in addition to mixing.

Various options for closing such passively-gassed culturing devices are known to a person skilled in the art, which differ in particular in terms of their gas permeability, type of gas exchange (diffusion and/or convection) and suitability as a sterile barrier, for example, screw closures with or without a gas-permeable membrane, aluminum buckles, aluminum foil, plastic film, adhesive or push-on gas-permeable membranes, cotton wool plugs, glass plugs, etc.

A disadvantage of the above-described culturing devices with passive gassing is the position and orientation of the device opening via which the gas exchange takes place. The movement of the liquid in the culturing device, induced by the shaking movement, causes a gas flow within the headspace of the device. As a result of the geometry of the device (in particular in flask-like devices), the large distance between the liquid causing the gas movement and the device opening, as well as the orientation of the opening surface parallel to the base of the device and thus parallel to the main movement surface of the liquid, the movement and thus the local mixing of the gas phase in the headspace directly in front of the device opening is very weak and additionally virtually free of pressure gradients with an appreciable vertical portion relative to the opening surface, such that both the gas exchange within the headspace close to the opening, and also the gas exchange with the ambient gas phase above the device opening, must take place predominantly by diffusion without an appreciable pressure gradient and is accordingly slow.

WO 001987007293A1 describes a shaking flask with means for improving gas exchange. This document also discloses chicanes for turbulent mixing of the headspace. While this solves the problem of poor mixing of the gas phase within the headspace, the above-described disadvantageous position, orientation and configuration of the gas exchange/filling opening nonetheless remain.

DE000004415444C2 and EP000000905229A2 describe devices for determining and monitoring the physiological state of microbial cultures using the oxygen transfer rate. This document also discloses methods and a device for actively gassing shaking flasks as culture device, with the gassing taking place via separate gas exchange openings which are open to the top. The active gassing solves the problem of slow gas exchange both within the headspace close to the opening and also with the ambient gas phase via the device openings, since the active gassing leads to efficient convective has exchange. However, the active gassing disadvantageously increasing the complexity and handleability of the entire culturing device, since the shaking flasks must be equipped with hoses, pumps, valves, etc.

Thus, no devices for the culturing of cells in a shaking operation are known which offer the advantages of passive gassing, in particular with regard to lower complexity and easier handleability and maintainability, with simultaneously optimized gas exchange between headspace and ambient gas phase as well as optimal gas mixing within the headspace.

### OBJECT OF THE INVENTION

The object of the present invention is therefore to provide a device by means of which the culturing of cells with passive gassing in a shaking operation can take place with optimal gas exchange between headspace and ambient gas phase and also with optimal gas mixing within the headspace. The object underlying the invention is solved by a device according to claim 1 and also a method for using the device according to claim 10; preferred configurations are given by the dependent claims and the description.

### SUMMARY OF THE INVENTION

The invention provides device for culturing cells in a shaking operation with passive gas exchange, which includes at least one gas exchange opening, characterized in that the opening surface of the gas exchange opening is not oriented parallel with the movement vector of the shaking movement at at least one point in time of the shaking movement.

In some embodiments, the gas exchange opening is closed with a gas-permeable sterile barrier. In some embodiments, at least two gas exchange openings are arranged opposite one another. In some embodiments, a plurality of gas exchange openings are arranged radially circumferentially and are only separated by connecting struts such that a virtually continuous circumferential gas exchange opening is formed. In some embodiments, at least one gas exchange opening is located at a height above the base of the device which is at least twice as high as the height of the unshaken culture liquid above the base when the device is filled to 5% to 20% of its nominal volume.

In some embodiments at least one further opening for handling liquids is provided, which opening is closed by a closure or a closure with integrated feeding reservoir.

In some embodiments, at least one anti-splash protrusion runs between the culture liquid and at least one gas exchange opening.

In some embodiments, a wall has the external geometry of a shaking flask.

In some embodiments, the angle α between the opening surface of at least one gas exchange opening and the movement vector is greater than 45° at at least one point in time of the shaking movement.

The invention also provides a method for culturing cells in a shaking operation with passive gas exchange, using the device described herein, characterized in that at least one movement vector of the shaking movement does not run parallel to the opening surface of at least one gas exchange opening.

### BRIEF DESCRIPTION OF THE DRAWINGS

FIG. 1: a schematic depiction of the device according to the invention.

FIG. 2: a schematic depiction of embodiments of the device according to the invention as a shaking flask with different gas exchange openings 4.

FIG. 3: a depiction of an embodiment of the invention as a shaking flask with circumferential gas exchange openings 4.

FIG. 4: a schematic depiction of the device according to the invention with a feeding system connected via the further opening 11.

### DETAILED DESCRIPTION OF THE INVENTION

Introduction to the Invention

The invention is particularly applicable to culturing processes with a high oxygen requirement or for cultures with high demands in terms of the formation of a gas phase equilibrium with the surroundings. The invention is advantageously applicable in culturing processes with passive gassing, the mixing of which is achieved by shaking; in particular, but not exclusively, in the field of shaking flask culturing.

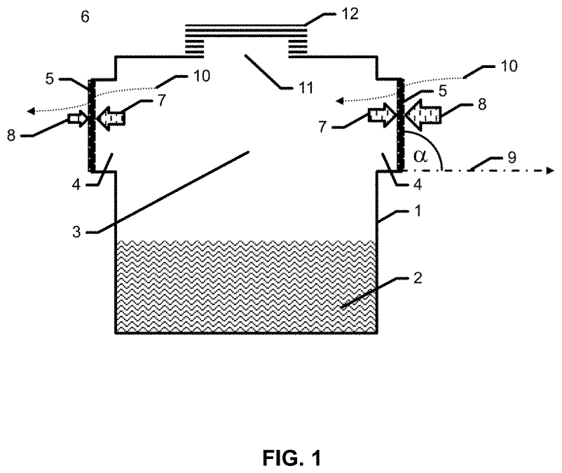

With reference to FIGS. 1-4, the culturing of cells takes place in a wide variety of devices which store and mix the culture liquid 2 as well as setting the culture conditions. Here, the composition of the gas phase, from which the culture liquid 2 is supplied with oxygen or via which physiologically relevant gases such as CO₂ are exchanged, is of particular significance.

Maintaining a particular gas phase composition is critical to the success of every culturing process. Thus, for example, only with a sufficiently high oxygen concentration in the gas phase can the oxygen partial pressure required for complete aerobic metabolism in the culture liquid 2 be maintained. Because of the low solubility of oxygen in aqueous phases and the continuous consumption of oxygen by respiring cells, a device for culturing cells must provide suitable means for continuous gas exchange 10.

At the same time, this gas exchange 10 must take place while maintaining sterility as much as possible, in order to prevent contamination of the culturing process with extraneous cells. As a result, the gas phase is commonly, and depending on the sterility requirements, separated in the culturing device from the external gas phase by a sterile barrier 5. While in actively-gassed culturing devices such as stirred-tank fermenters, sterile-filtered gas mixtures are actively introduced into the culture liquid 2 (e.g. by means of pressurized air as a bubble column), gas exchange 10 in passively-gassed culturing devices such as shaking flasks only takes place by slow diffusion and convection between the headspace 3 of the culturing device and the ambient gas phase 6 thereof. In passively-gassed devices, in particular in the case of culturing rapidly-growing aerobic organisms, this may therefore lead to oxygen limitations, which slow down the culturing process and also negatively impact the formation of undesired metabolites or fermentation products. This effect is increased in some applications by installations or additions to the culturing device, in particular sensors and probes and also feeding and sampling systems, which hinder the diffusion and convection both between and within the headspace gas phase 3 of the culturing device and the ambient gas phase 6.

To ensure clarity regarding some of the terms used in the description, these terms will be defined and explained below and in the course of the description.

Gas exchange 10 denotes the transition of gaseous substances between two phases, in particular between the ambient gas phase 6 and the headspace gas phase 3, and also between the headspace gas phase 3 and the culture liquid 2. Gas exchange 10 occurs through the movement of the substances to be exchanged, in particular by convection or diffusion. In active gas exchange 10, this occurs by means of gas conveying devices and methods provided for this purpose, in particular by pumps or overpressure systems, with the gas to be exchanged customarily being guided into the device or the culture liquid 2 through pipelines or hoses. Active gas exchange 10 can for example be directly regulated by changing the volumetric flow via the speed of the pump. In passive gas exchange 10, this occurs without gas conveying devices and methods provided for this purpose, but rather by utilizing given states, such as ambient pressure and the construction and movement of the device for culturing cells. Passive gas exchange 10 cannot be directly regulated but rather only by changing the given states; in particular, by not exclusively, by changing the temperature, the pressure or the composition of the ambient gas phase 6 and also by changing the construction or the movement of the device for culturing cells.

The headspace 3 comprises the inner volume of the device which is not filled with culture liquid 2. According to the invention, the headspace contains the headspace gas phase 3, to both of which reference will be made, either jointly or separately, as headspace with headspace gas phase 3.

The device according to the invention is located in a surroundings with an ambient gas phase 6, to both of which reference will be made, either jointly or separately, as surroundings with ambient gas phase 6. The composition of the ambient gas phase 6 corresponds either to natural ambient air or is defined in a targeted manner, for example by gas mixing stations.

The use according to the invention of the device occurs in a shaking operation, in particular, but not exclusively, by rocking, tumbling or orbital shakers. According to the invention, the shaking operation can take place both continuously or with interruptions. Because of the shaking movement, the device has a relative speed at least relative to the surroundings with ambient gas phase 6 along a speed vector, which is referred to here as movement vector 9. Because of accelerations in the course of the shaking movement, the movement vector 9 may change over time, in particular, but not exclusively, in terms of direction and magnitude. Because of the relative movement of the device relative to the surroundings with ambient gas phase 6, caused by the shaking, gas flows around the device, such that the pressure on the device changes depending on the point of the device being considered, and a local ambient pressure 8 is set up for each point of the device being flowed around, which ambient pressure can change with the shaking movement and the movement vector 9. The same applies for the movement of the headspace gas phase in the headspace 3, such that here, too, a local headspace pressure 7 arises for each considered point of the device being flowed around. At openings of the device which are not closed or gas permeable, in particular, but not exclusively, at gas exchange openings 4, the difference or pressure gradient between headspace pressure 7 and ambient pressure 8 can give rise to a convective or diffusive pressure compensation via at least one gas exchange 10. This all applies both to total pressure and also partial pressure of individual components of a phase.

Culture liquids 2, liquids or liquid phases for the purposes of the invention are pure or mixed substances which are not gases and have fluid properties. Liquids for the purposes of the invention are thus in particular, but not exclusively, liquid pure substances, solutions, emulsions, dispersions, slurries, suspensions and foams. Culture liquids 2 consist in particular, but not exclusively, of nutrient medium and cells.

A gas exchange opening 4 is an opening in the wall 1 of the device, which primarily serves the purpose of gas exchange 10. It is characterized by an opening surface which is the smallest possible area suitable for closing the opening. The opening surface can be curved, and can be described by at least one normal vector at any point. The orientation of an opening or opening surface relative to the movement vector 9 of the shaking movement is described by the angle α which is the angle of intersection between the movement vector b (9) and a normal vector n on the opening surface, giving

$$α = arcsin \frac{\vert n \cdot b \vert}{\vert n \vert \cdot \vert b \vert}$$

Depending on the curvature of the opening surface, the angle α can differ from point to point, since the normal vectors of a curved surface are not all parallel.

The opening surface of a gas exchange opening 4 and a movement vector 9 are classed as not being arranged parallel if, for at least one point on the opening surface of the gas exchange opening 4 considered, the angle α is greater than 0°.

A sterile barrier 5 is a gas-permeable device which is used in particular to prevent, reduce or completely stop the penetration of undesired cells, viruses or other impurities into the interior of the device according to the invention through at least one gas exchange opening 4 or further opening 11. Sterile barriers 5 according to the invention allow at least one gas exchange 10 between the headspace with headspace gas phase 3 and the surroundings with ambient gas phase 6, in particular via diffusion and/or convection. Sterile barriers 5 for the purposes of the invention are in particular, but not exclusively, sterile filters, porous membranes (e.g. PTFE, cellulose, hydrophilic or hydrophobic, etc.), cotton wool plugs or pads and also open-pore foams made of silicone, polyurethane or other plastics. Sterile barriers 5 for the purposes of the invention are connected to the wall 1 of the device according to the invention, in particular, but not exclusively, by direct adhesive bonding or welding, and also indirectly by suitable closure systems with screw or snap-on closures or any positive-locking or frictional connections.

Solution

The object is solved according to the invention by a device for culturing cells in a shaking operation, wherein the claimed device has at least one gas exchange opening 4, the opening surface of which is not oriented parallel to the movement vector 9 of the shaking movement. Brought about by the shaking movement and the associated flowing of the ambient gas phase 6 around the outside of the device and the headspace gas phase 3 around the inside of the device, this arrangement leads to the formation of a pressure gradient between the headspace gas phase 3 and the ambient gas phase 6 which changes depending on the shaking movement and which advantageously influences the gas exchange between headspace gas phase 3 and ambient gas phase 6 as well as the mixing of the gas within the headspace 3.

If the surroundings 6 and the headspace 3 are convectively connected, this pressure gradient brings about a compensation flow, which, depending on the direction of the pressure gradient, leads to the flowing of the gas from the surroundings 6 into the headspace 3 of the device or to the flowing of the gas from the headspace 3 of the device out into the surroundings 6. In the case of periodic shaking movements, an oscillating breathing-like movement of gas between headspace 3 and surroundings 6 thus arises, which on the one hand enables a significantly more efficient gas exchange 10 than the passively-gassed culturing devices known from the prior art and on the other hand improves the mixing within the headspace 3.

If the surroundings 6 and the headspace 3 are convectively separated from one another, but diffusively connected to one another, then the difference between the local ambient pressure 8 and/or headspace pressure 7 caused by the shaking movement is accompanied by an increase in the speed of diffusion between the ambient gas phase 6 and the headspace gas phase 3. In the case of periodic shaking movements, oscillating diffusion speeds and directions thus arise, which on the one hand enable a more efficient gas exchange 10 than the passively-gassed culturing devices known from the prior art and on the other hand improve the mixing within the headspace 3.

According to the invention, the pressure gradient between surroundings 6 and headspace 3 is formed in particular along the movement vector 9 of the device according to the invention in a shaking operation. In this respect, the pressure gradient increases for the same overall surface area of the gas exchange opening 4 with an increasing angle α between the opening surface thereof and the movement vector 9 of 0° to 90°. With the stated overall surface area of the gas exchange opening 4, the achievable pressure gradient is at its maximum when the movement vector 9 is orthogonal to the opening surface and at its minimum when the movement vector 9 is parallel to the opening surface, with the latter arrangement corresponding to the current state of the art.

The device is advantageously used in a method according to the invention for culturing cells in a shaking operation. To this end, a device according to the invention is filled with culture liquid 2. A headspace gas phase 3 is formed in the headspace via at least one gas exchange opening 4 and/or at least one further opening 11. The filled device according to the invention is placed on or in a shaker and thus within surroundings with an ambient gas phase 6. In the shaking operation, cells are now cultured in the device according to the invention, with the shaking operation being characterized in that at least one movement vector 9 of the shaking movement does not run parallel to the opening surface of at least one gas exchange opening 4. The method according to the invention thereby ensures that the device according to the invention is used under such surrounding conditions that lead to the formation of at least one pressure gradient between the headspace gas phase 3 of the device according to the invention and the ambient gas phase 6 thereof, which pressure gradient changes depending on the shaking movement, and the gas exchange 10 between headspace gas phase 3 and ambient gas phase 6, as well as the gas mixing within the headspace gas phase 3, are advantageously improved compared to the prior art.

In an advantageous configuration of the invention, the angle α between the opening surface of at least one gas exchange opening 4 and at least one movement vector 9 is 90°. In further advantageous configurations of the invention, the angle α between the opening surface of at least one gas exchange opening 4 and at least one movement vector 9 is 60°≤α≤90° or 45°≤α≤90° or 45°≤α≤60° or 30°≤α≤90° or 30°≤α≤60° or 30°≤α≤45° or 0°≤α or 15°≤α or 30°≤α or 45°≤α or 60°≤α.

In an advantageous configuration of the invention, the device comprises a plurality of gas exchange openings 4, in particular, but not exclusively, two, three, four, five or six.

In an advantageous configuration of the invention with a plurality of gas exchange openings 4, these are arranged such that for every one, in at least one state of the shaking movement, at least one of the above-mentioned conditions is met for the angle α between the opening surface thereof and at least one movement vector 9.

In an advantageous configuration of the invention, the device is surrounded by a plurality of gas exchange openings 4 such that for virtually every state of the shaking movement, at least one of the above-mentioned conditions for the angle α between opening surface and movement vector 9 is met for at least one of these gas exchange openings 4.

In an advantageous configuration of the invention, each gas exchange opening 4 of the device according to the invention is closed with a sterile barrier 5 which hinders or completely prevents the penetration of extraneous cells but which is permeable to the gas phase components that are to be exchanged. In an advantageous configuration of the invention, at least one sterile barrier 5 allows the gas exchange 10 via convection and diffusion.

In an advantageous configuration of the invention, the gas exchange openings 4 are arranged in a shared plane, in order to ensure the stability of the device during the shaking.

In an advantageous configuration of the invention, the device can be autoclaved or is already pre-sterilized. In some embodiments of the invention, the sterile barriers 5 can be replaced.

In an advantageous configuration of the invention, the device comprises, aside from at least one gas exchange opening 4, at least one further opening 11, which is in particular, but not exclusively, suitable for filling and emptying the device, drawing off samples, attaching probes and sensors or attaching feeding and dispensing devices. In an advantageous configuration of the invention, such further openings 11 comprise a closure system for attaching external devices or closures 12, in particular, but not exclusively, closure systems with buckle, latch, form-fitting or screw mechanisms.

In an advantageous configuration of the invention, one or more gas exchange openings 4 are set away from the wall 1 of the device according to the invention and/or have an inner anti-splash protrusion 13 placed under them, in order to prevent liquid escaping as a result of shaking or handling movements and also to prevent the liquid from coming into contact with sterile barriers 5 which may be present. In some embodiments of the invention, one or more gas exchange openings 4 are placed at a height above the base of the device which is at least twice as high as the height of the unshaken culture liquid 2 above the base with the usual amount of filling, in particular, but not exclusively, when the device is filled to 5% to 20% of its nominal volume.

In some embodiments, the gas exchange 10 takes place between the headspace gas phase 3 and the ambient gas phase 6 unidirectionally, in other embodiments it takes place bidirectionally.

In some embodiments of the device according to the invention, selective sterile barriers 5 are used, which enable the gas exchange 10 of only one or several specific gas phase components via the gas exchange opening 4 in question.

In some embodiments of the device according to the invention, the wall 1 is made of glass. In other embodiments of the device according to the invention, the wall 1 is produced from plastic, in particular, but not exclusively, by means of injection molding or centrifugal casting.

The present invention will be described in more detail using the figures and embodiment examples. Reference signs in the figures indicating components of the invention which have already been used in the same figure or in another figure under the same circumstances or in the same depiction will sometimes be omitted in order to maintain the clarity and intelligibility of the figures. Graphic elements without reference signs must therefore be interpreted taking into account the list of reference signs, the other figures, the indicated depictions within the same figure, the patterning or structuring of graphic elements that have already been indicated, and with reference to the entire description and the claims.

Preferred Embodiments

FIG. 1 shows a schematic depiction of the device according to the invention. The device includes at least one wall 1 which encloses the interior of the device according to the invention. Culture liquid 2 and, above it, the headspace with headspace gas phase 3, are located in the interior of the device. The headspace gas phase 3 is connected to the surroundings with surrounding gas phase 6 via at least one gas exchange opening 4, such that at least one gas exchange 10 between the headspace gas phase 3 and the ambient gas phase 6 can take place in particular, but not exclusively, via diffusion and/or convection. Each gas exchange opening 4 can be closed via a gas-permeable sterile barrier 5. During the culturing of cells, the device according to the invention is mixed by occasional, periodic or continuous shaking, with the shaking movement being characterized by at least one movement vector 9 at each point in time. As a result of the shaking movement, a pressure gradient between local headspace pressure 7 and local ambient pressure 8 builds up via a gas exchange opening 4 and in particular via a sterile barrier 5 depending on the angle α between the opening surface of the gas exchange opening 4 and the movement vector 9, which pressure gradient increases the convective gas exchange 10 through compensation flow and the diffusive gas exchange 10 through increasing the concentration difference between headspace 3 and surroundings 6. In an advantageous configuration of the invention, the device additionally comprises at least one further opening 11 which is closed at least during the culturing of cells, in particular, but not exclusively, by a suitable closure 12.

Depending on the arrangement of the gas exchange openings 4, in the case of an embodiment of the device according to the invention having a plurality of gas exchange openings 4, different pressure gradients form in at least one state of movement with a given movement vector 9 via different gas exchange openings 4 and/or sterile barriers 5. As illustrated by way of example in FIG. 1, this leads, on the inflow side of the device (FIG. 1, right-hand side), to a higher local ambient pressure 8 compared to the local headspace pressure 7, such that, at this gas exchange opening 4, ambient gas phase components predominantly flow into (10, right-hand side) the headspace 3 of the device, while on the opposite side, exactly the converse pressure gradient arises and here (FIG. 1, left-hand gas exchange opening 4) headspace gas phase components predominantly flow out of (10, left-hand side) the device into the surroundings 6. This gives rise on the one hand to an advantageous increase in the total gas exchange 10 between headspace gas phase 3 and ambient gas phase 6 and on the other hand these different pressure gradients give rise to improved mixing of the headspace gas phase 3 as a result of an additional cross-diffusion/convection within the headspace 3 and also the interaction thereof with the gas phase movement which is brought about by the movement of the device and the culture liquid 2.

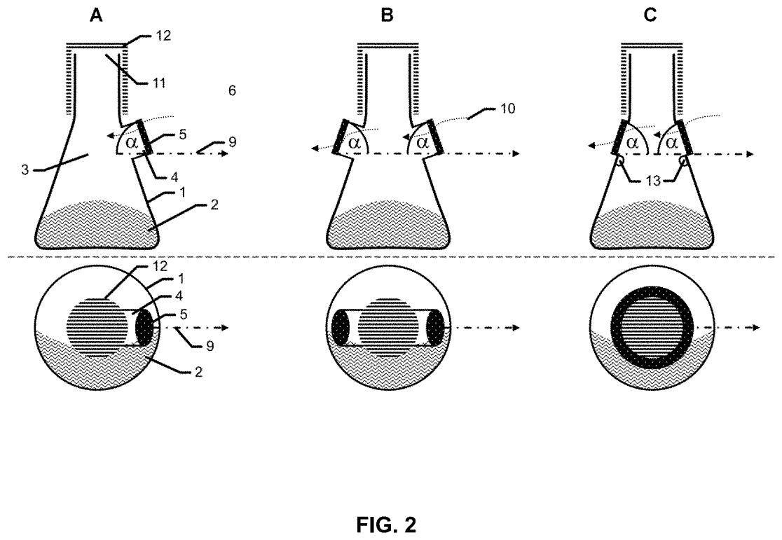

FIG. 2 shows a schematic depiction of three embodiments of the device according to the invention as a shaking flask with different gas exchange openings 4. A side view (above the dashed line) and a plan view (below the dashed line) are given for each embodiment (A, B, C). The embodiments in FIG. 2 A-C all comprise a further opening 11 for filling, emptying, drawing off samples, feeding or performing various interventions during the culturing process, which is configured here as a conventional flask opening and is closed with a cap as closure 12.

The embodiment of the device according to the invention as a shaking flask comprises, with typical nominal filling volumes of up to 30%, a comparatively large, upwardly tapering headspace with headspace gas phase 3. This conventional headspace geometry makes use, in particular in orbitally-shaken operation, of the advantages of the passive gas exchange 10 according to the invention via gas exchange openings 4, the opening surface of which is not oriented parallel to the movement vector 9 of the shaking movement. In contrast thereto, shaking flasks according to the prior only have a single upward opening, which is only poorly suited for the gas exchange 10 because of its orientation which is entirely or, during shaking operation, predominantly, parallel to the shaking movement.

In shaking flasks according to the prior art, this is increased by tapering the flask toward the opening, such that, in orbital shaking operation, a rotating gas vortex arises in the headspace 3 of the flask, the movement vectors of which also span a surface which runs disadvantageously parallel to the single opening of the shaking flask according to the prior art, such that no pressure gradient arises and can oscillate between local headpsace pressure 7 and local ambient pressure 8 via the opening of the shaking flask as a device according to the prior art, which pressure gradient is advantageous according to the invention and which promotes the gas exchange 10 as well as the mixing.

FIG. 2A shows an embodiment of the device according to the invention as a shaking flask with a lateral gas exchange opening 4, the opening surface of which, in the state of movement depicted, is oriented at an angle of α=71° to the movement vector 9. Compared to the entirely orthogonal (α=90°) arrangement of FIG. 1, the gas exchange 10 here may be somewhat weaker, but it is still considerably better than in devices with passive gassing according to the current state of the art. It is advantageous here, as is also the case in FIG. 2B, to obliquely set the gas exchange opening 4 away from the wall 1 of the device according to the invention, such that splashes and condensation liquid cannot disadvantageously wet or close off the sterile barrier 5, and instead flows back into the culture liquid 2.

FIG. 2B shows an embodiment of the device according to the invention as a shaking flask with two lateral gas exchange openings 4, the opening surfaces of which, in the state of movement depicted, are oriented at an angle of α=71° to the movement vector 9. Similarly to the embodiment example of FIG. 1, the gas exchange openings 4 are here arranged opposite one another, such that better mixing within the headspace gas phase 3 is set up by crossflow.

FIG. 2C shows an embodiment of the device according to the invention as a shaking flask with a plurality of circumferential gas exchange openings 4 which are all provided with a shared sterile barrier 5 and thus form a virtually continuous gas exchange opening 4. This embodiment is especially advantageous in the orbital shaking operation of the device according to the invention, since it ensures that the nonparallel arrangement between movement vector 9 and the opening surface of the gas exchange opening 4, which arrangement promotes the gas exchange 10, is present at any point in time of the shaking movement. Moreover, the embodiment depicted in FIG. 2C enables, by the radial arrangement of the virtually continuous circumferential gas opening 4, the generation of a gas exchange (10) vortex that rotates with the shaking movement, since with orbital shaking operation, the opposite surfaces/points of the maximum pressure gradient move once around the entire flask via the gas exchange opening 4 and the sterile barrier 5 within one period of the orbital shaking movement, due to the permanently changing movement vector 9.

In contrast to FIG. 2A and 2 B, in the embodiment of FIG. 2C, the gas exchange openings 4 are not set away, but rather directly integrated into the wall 1, such that the sterile barrier 5 lies directly on the wall 1 of the device and the angle α between the opening surface of the gas exchange opening 4 and the movement vector 9 arises through the geometry of the wall 1. This affords advantages in particular in terms of simplifying the production of a device according to the invention, since here the sterile barrier 5 can be more easily applied, in particular, but not exclusively, by adhesive bonding or ultrasonic welding to the wall 1. It is further advantageous that, by not setting the gas exchange openings 4 away, the center of gravity of the device according to the invention remains closer to the base, which, particularly with rapid shaking, has a positive effect on the stability of the device and also on the maximum permitted shaking speed. In order to prevent wetting of the non-set-away sterile barriers 5 by spray, the embodiment of FIG. 2C comprises an anti-splash protrusion 13 which is fitted between gas exchange opening 4 and culture liquid 2 in order to retain splashing liquid. An anti-splash protrusion 13 can also be used advantageously above the gas exchange opening 4, in particular in order to prevent wetting of the sterile barrier 5 with condensed liquid flowing out of the upper part of the device. In some embodiments, the setting away of at least one gas exchange opening 4 is combined with at least one of the above-mentioned anti-splash protrusions 13.

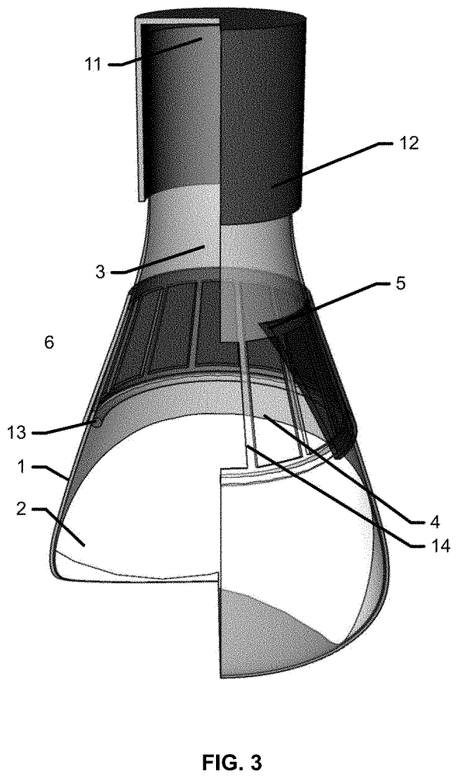

A selected implementation of the device according to the invention from FIG. 2C is depicted as a perspective partial section in FIG. 3. The device is designed here as a conventional shaking flask comprising a wall 1 in which a plurality of radially circumferential gas exchange openings 4 are made above the culture liquid 2, such that an entire virtually continuous gas exchange opening 4 is formed for the gas exchange 10 between headspace gas phase 3 and ambient gas phase 6, which opening is only interrupted by narrow connecting struts 14. Also circumferentially, a contiguous sterile barrier 5 (shown here in truncated form) covers all gas exchange openings 4 from the outside. An anti-splash protrusion 13 runs annularly below this barrier. The device also has a further opening 11 with closure 12.

The embodiment illustrated in FIG. 3 of the device according to the invention is particularly suitable for being produced from plastic as a disposable product and delivered pre-sterilized.

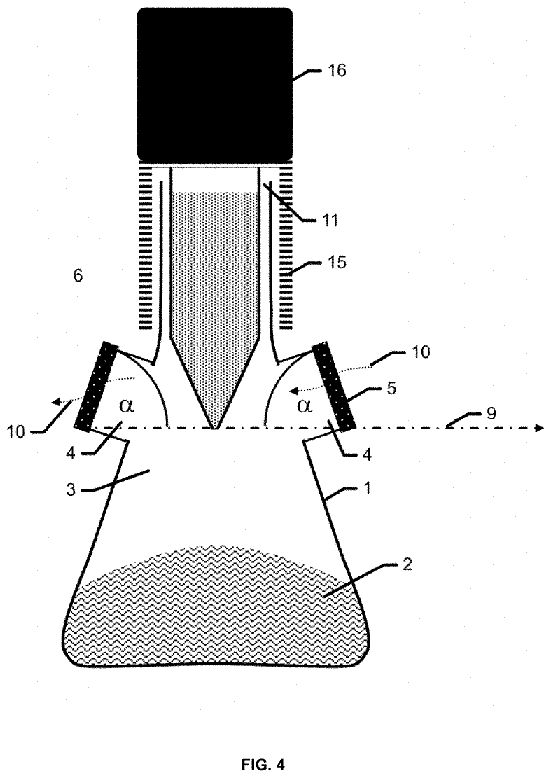

FIG. 4 shows a schematic depiction of the device according to the invention with a feeding system connected via a further opening 11. The device is designed here as a conventional shaking flask, comprising a wall 1 on which a plurality of gas exchange openings 4 are made above the culture liquid 2 in the set-away configuration for gas exchange 10 between headspace gas phase 3 and ambient gas phase 6 such that, at at least one point in time of the shaking movement, the angle α between movement vector 9 and the opening surface of at least one gas exchange opening 4 is greater than 0° and movement vector 9 and opening surface of at least one gas exchange opening 4 are not oriented parallel to one another. A respective gas-permeable sterile barrier 5 covers each gas exchange opening 4. The device has a further opening 11, which, during the operation of the device for culturing cells according to the invention, is closed by a closure with integrated feeding reservoir 15 and to which a metering system 16 is fitted. This advantageously enables the addition of substrates and nutrients during the culturing of cells, in particular, but not exclusively, as a fed-batch process, such that the device depicted here, as a result of the improved gas exchange 10 according to the invention, is able to carry out cell culturing processes even with passive gassing without any oxygen limitation, which cell culturing processes would otherwise only be able to be carried out in considerably more complex actively-gassed stirred-tank fermenters.

### LIST OF REFERENCE SIGNS

For the respective interpretation of the reference signs, attention should be paid to the description and the claims.
1 Wall
2 Culture liquid
3 Headspace with headspace gas phase
4 Gas exchange opening
5 Sterile barrier
6 Surroundings with ambient gas phase
7 Local headspace pressure
8 Local ambient pressure
9 Movement vector
10 Gas exchange
11 Further opening
12 Closure
13 Anti-splash protrusion
14 Connecting struts
15 Closure with integrated feeding reservoir
16 Metering system

1. A device for culturing cells during a shaking operation with passive gas exchange, the device comprising at least one gas exchange opening, wherein an opening surface of the gas exchange opening is not oriented parallel with a movement vector of a shaking movement at at least one point in time of the shaking movement.

2. The device according to claim 1, wherein at least one gas exchange opening is closed with a gas-permeable sterile barrier.

3. The device according to claim 1, wherein at least two gas exchange openings are arranged opposite one another.

4. The device according to claim 1, wherein a plurality of gas exchange openings are arranged radially circumferentially and are only separated by connecting struts such that a virtually continuous circumferential gas exchange opening is formed.

5. The device according to claim 1, wherein at least one further opening for handling liquids is provided, which opening is closed by a closure or a closure with integrated feeding reservoir.

6. The device according to claim 1, wherein at least one anti-splash protrusion runs between the culture liquid and at least one gas exchange opening.

7. The device according to claim 1, wherein a wall has an external geometry of a shaking flask.

8. The device according to claim 1, wherein an angle α between the opening surface of at least one gas exchange opening and the movement vector is greater than 45° at at least one point in time of the shaking movement.

9. The device according to claim 1, wherein at least one gas exchange opening is located at a height above the base of the device which is at least twice as high as the height of the unshaken culture liquid above the base when the device is filled to 5% to 20% of its nominal volume.

10. A method for culturing cells in a shaking operation with passive gas exchange, using the device according to claim 1, wherein at least one movement vector of the shaking movement does not run parallel to the opening surface of at least one gas exchange opening.
